(19) United States
(12) Patent Application Publication
(10) Pub. No.: US 2015/0260421 A1
(43) Pub. Date: Sep. 17, 2015

(54) AIR TREATMENT APPARATUS

(71) Applicant: WLI Trading Ltd, Dublin (IE)

(72) Inventors: Jonathan Ben-David, Qingdao (CN); Heung Soon Kim, Qingdao (CN)

(21) Appl. No.: 14/433,338

(22) PCT Filed: Sep. 25, 2013
(86) PCT No.: PCT/IB2013/058854

§ 371 Date: Apr. 2, 2015

(30) Foreign Application Priority Data
Oct. 3, 2012 (GB) 1217644.2

(51) Int. Cl. F24F 3/16; F24F 3/14; A61L 9/20

(52) U.S. Cl. CPC F24F 3/16; A61L 9/20; F24F 3/14; F24F 2003/1667

(57) ABSTRACT

Air treatment apparatus including means for causing ultraviolet radiation to be applied to fluid or moisture which is to be discharged from the apparatus.

This invention relates to air treatment apparatus. In particular, it relates to air treatment apparatus such as humidifiers, de-humidifiers, air conditioners or any other similar types of apparatus which are commonly used in households, offices, business premises, shops and other establishments and used to treat air by altering its moisture content, its climate and so on.

Devices of this type are commonly used to add moisture/increase humidity or to remove moisture from an environment. Devices such as humidifiers which add water often work on ultrasonic or other principles. They generally utilise a tank of water which is acted upon by a heater, pump, ultrasonic vibrator or other means in order to cause a stream of water droplets with air that exit an outlet of the humidifier in the form of a fog. This may be cooled. De-humidifiers and air conditioners essentially work in the other direction by drawing moisture laden air in and they cause moisture to be outputted through an outlet positioned behind the device, via a tube to the exterior or to an extraction system.

There is a problem with such air treatment devices, particularly, but no exclusively, with the ones which utilise a tank of water, in that if the water is kept for any length of time bacteria, mould or other contaminants may tend to multiply in it. If the water is then ejected into a room in the form of small droplets, these may also be contaminated and this can lead to possible health hazards. The problem is exacerbated when, as is often the case, such devices are used in very warm environments. In addition, if the actual outlet itself is contacted by human fingers there is an additional risk of contamination by bacteria or other infection.

The present invention arose in an attempt to provide an improved air treatment apparatus.

According to the present invention there is provided air treatment apparatus including means for causing ultraviolet radiation to be applied to fluid or moisture which is to be discharged from the apparatus.

Preferably, the apparatus is a humidifier, de-humidifier or air conditioning apparatus.

The apparatus may comprise a conduit or vessel adapted to receive moisture and which is of UV transmissive material and positioned so that part of it is directly acted upon by a UV generated by a UV generation means.

A conduit may be partially or wholly wrapped around a UV generator.

A housing may be provided comprising a body of UV reflecting material provided around the conduit or vessel so as to reflect radiation back thereto.

The housing may further comprise a conduit formed of a UV transmissive material, having an inlet and an outlet and positioned so that part of it is wrapped around at least part of the UV source and further comprising reflective means for causing UV radiation to be reflected onto one or more parts of the conduit which extends beyond the part which is wrapped around the UV source.

The reflective means may comprise an end part through which at least the outlet passes and which has a surface which reflects UV radiation.

Embodiments of the invention will now be described, by way of example only, with reference to the accompanying drawings, in which:

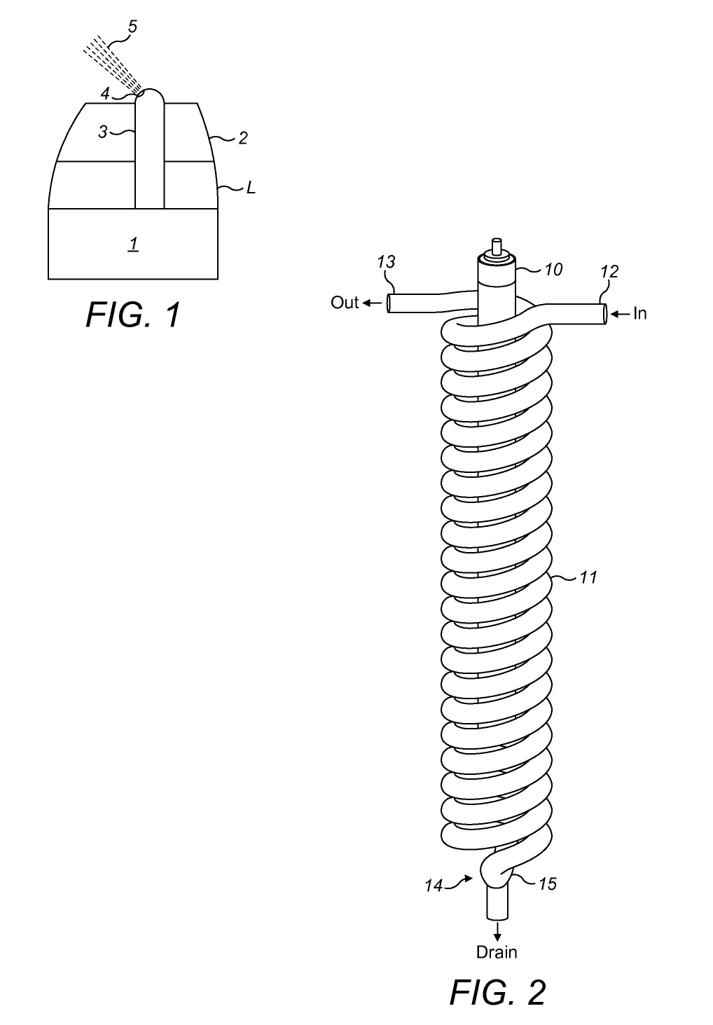

FIG. 1 shows schematically a humidifier;

FIG. 2 shows a part of a humidifier for sterilising discharge moisture with UV;

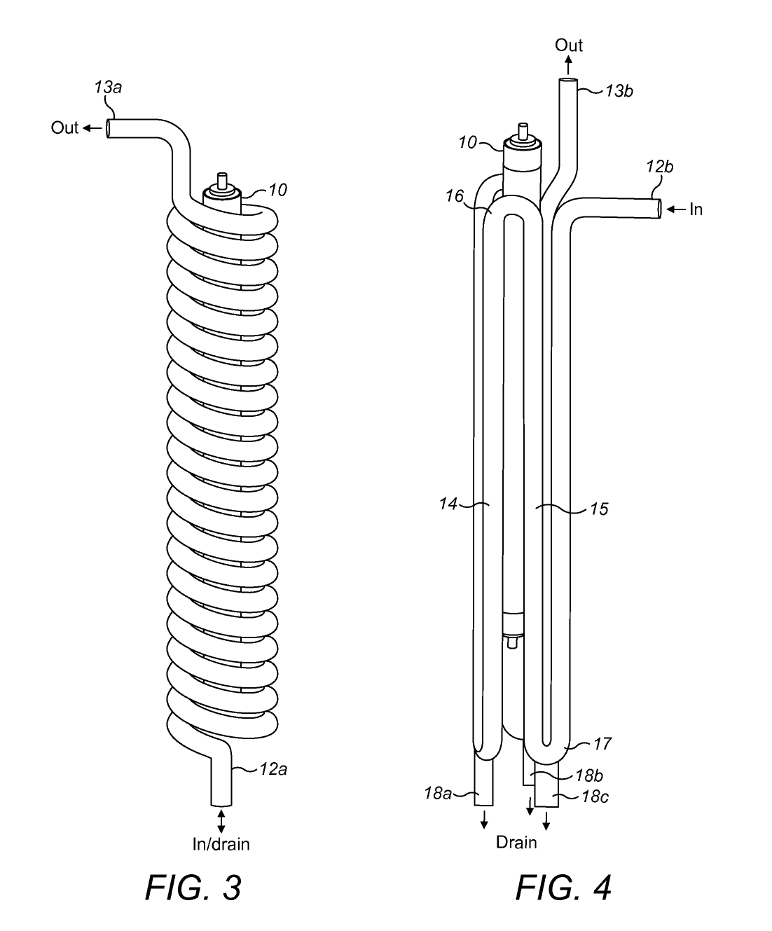

FIGS. 3 to 6 show alternative embodiments;

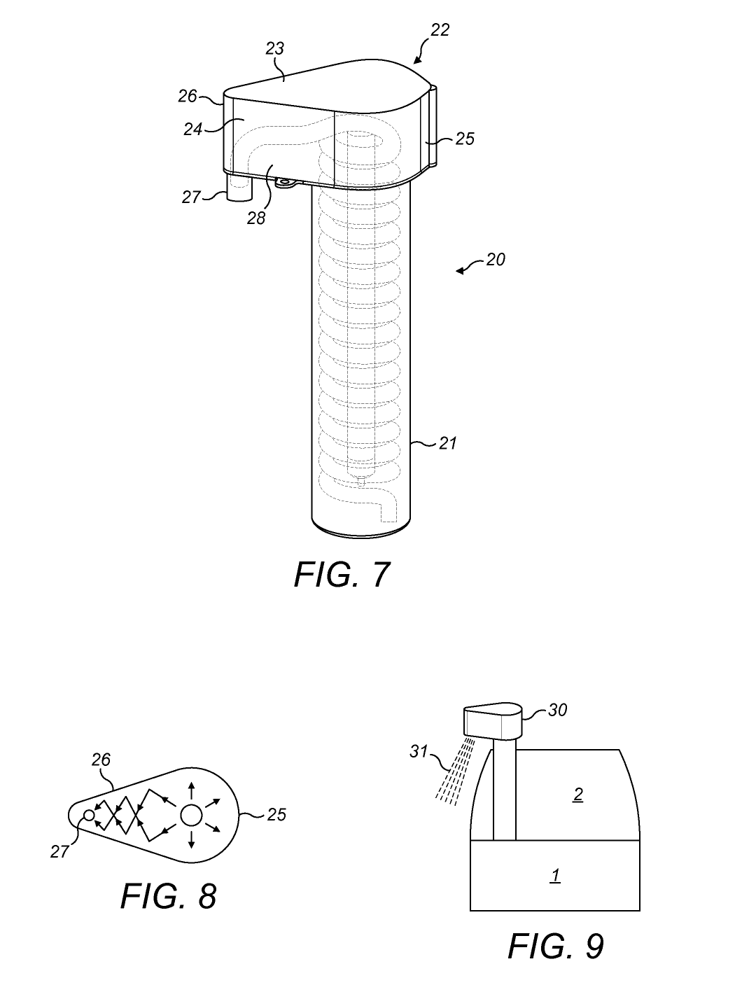

FIG. 7 shows schematically a reflective housing and conduit;

FIG. 8 is an explanatory diagram showing schematically a cross-sectional end view of a housing; and

FIG. 9 shows a humidifier with a UV sterilising means.

Referring to the figures, FIG. 1 shows schematically a humidifier. This comprises a body having electric controls (not shown) and a motor driving an ultrasonic element (not shown but the functioning of which is well known in the art). This is shown at 1. A tank 2 is mounted on this and is filled with water, typically to a level L. The water within the tank is drawn into the body 1 and is acted upon by the ultrasonic generator to cause a mixture of water droplets and air to be entrained into an outlet pipe 3 from which it is discharged through an orifice 4 into the atmosphere in the form of a stream of air and moisture 5. Such devices are generally fairly compact and may be placed upon a desk or other surface for example.

Note that instead of ultrasonic means, other means for generating a stream of moisture (ie air plus water droplets) may be used. The discharged moisture may also be cooled.

In embodiments of the invention, means are provided for generating ultraviolet (UV) radiation. UV radiation, as is well known, is effective at destroying bacteria and other microorganisms and contaminants. Any suitable UV source may be placed at a position where it can either act upon water within the tank itself or, more preferably, moisture within the discharge tube 3. UV lamps for sterilisation purposes are known in themselves and require a source of power. In some embodiments, a UV lamp (or two or more UV lamps) is provided within the discharge tube 3 itself and thus it provides sterilisation towards the actual discharge point of the moisture.

Note that hereinafter the term ‘moisture’ will be used to describe an air/water droplet mix (or a mix of air and any liquid) which may be used. Such a mix is eventually discharged from the apparatus.

FIGS. 2 to 6 show some non-limiting examples of parts of the apparatus which pass moisture across a UV lamp or other UV source.

Referring to FIG. 2, an elongate UV lamp 10 is mounted axially within a helical part of a conduit 11, which conduit includes an inlet 12 and an outlet 13. At least part of the conduit is wrapped helically around the lamp (but preferably spaced apart from it) as shown. The conduit is formed of a UV transmissive material, for example quartz, other glass or any other type of material which is transmissive to UV types of radiation such that UV radiation from the lamp 10 can pass into liquid or moisture within the conduit and out the opposite wall.

In the embodiment of FIG. 2, the inlet 12 and outlet 13 are both positioned towards the top (the top being relative to the device, as shown in FIG. 1, where this in effect replaces the outlet tube 3 and is therefore arranged to have a generally vertical disposition, which need not be precisely vertical).

Thus, the conduit extends from an inlet 12, wraps around the lamp 10 generally downwardly until a lowermost part 14 and then starts to wrap around in an upwardly direction up to the outlet 13. Thus, the ‘in’ parts and ‘out’ parts (ie upstream and downstream of the lower part 14) might be considered as being ‘interleaved’ in the helix (ie adjacent coils form part of, respectively, the upstream and downstream paths). The conduit may be formed of one single coiled conduit. At the bottom 14, a drain 15 is provided by providing a T-piece. In some embodiments, this might be omitted. The drain is very useful if any water condenses within the tube and forms liquid water droplets which might otherwise cause blockages or not be able to be omitted as a fog from the outlet 13 and such condensed droplets will simply trickle down the tube and out of the drain where they can be recycled back into the water tank 2.

The actual spacing of the helical part of the tube from the lamp will be determined by the power and type of the tube and will be such that efficient sterilising and destruction or rendering ineffective of bacteria is achieved, as will be known to those skilled in the art. Thus, moisture which has been generated by the ultrasonic action of the humidifier is applied to the inlet 12, through the conduit where it is sterilised by UV light from the UV lamp and out through outlet 13 where it is discharged as a stream or fog of moisture for humidifying an atmosphere around the device. The device will therefore be placed within a water tank.

The device will preferably be placed within a housing of UV reflective material, as will be described further below. Indeed, any of the embodiments described may be provided within a housing.

FIG. 3 shows an alternative embodiment in which the inlet 12a is provided towards the bottom and the outlet 13a is again at the top. In such a system, no specific drain, whether by T-piece or otherwise, is needed since any condensed water will simply flow downwards as drained liquid through the inlet, where it can be discarded, recycled back into the tank or otherwise utilised.

FIG. 4 shows an embodiment in which the conduit is provided in alternate substantially vertical folds laid in a generally elongate manner adjacent to, and generally parallel to, the elongate axis of the lamp 10. This embodiment includes an inlet 12b at the top and an outlet 13b at the top. Alternatively, the inlet may be provided at the bottom in a similar way to that of FIG. 3. Thus, instead of a helical wrapping of the conduit around the tube, the conduit is wrapped in alternate upwardly and downwardly directed folds 14, 15, for example, in the same way as the designs of radiators or other sinks. These are connected at their top and bottom ends by lateral parts of the conduit 16, 17, etc.

The conduit will generally be formed from a single tube.

Note, however, that in embodiments of the invention the conduit need not be formed of a single tube but can be formed of two or more tubes which are axially connected. However, it is much more efficient to make them of a single piece of glass, quartz, or otherwise.

The embodiment of FIG. 4 also includes one or more drains 8, again formed by T-pieces for example. In this case, since there are several ‘lower-most’ parts (eg lateral parts 16, 17, etc), it will often be found useful to include a plurality of drains, one provided at each lower-most fold of the conduit and these are shown by drains 18a to 18c in the figure. The number three of these is only for illustration only and may be more or less than this.

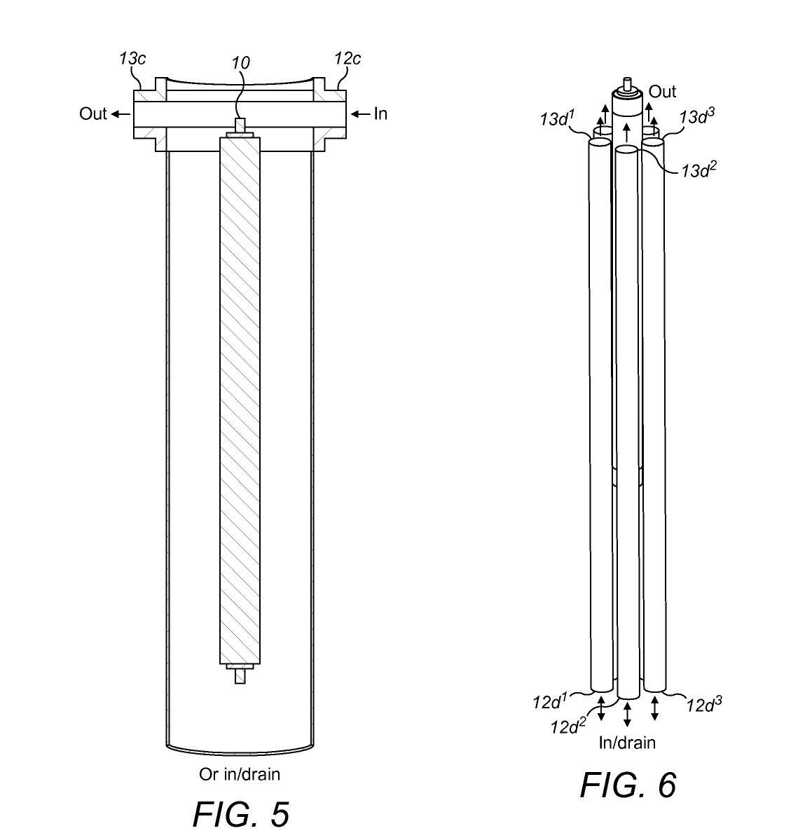

FIG. 5 shows an embodiment in which instead of a conduit which is wrapped (whether helically or otherwise) at least partially around the lamp, this simply comprises a vessel having an inlet 12c and an outlet 13c (shown in the top in this example although the inlet may again be at the bottom). Moisture enters at inlet 12c to the vessel where it directly contacts or is acted upon by UV generated from the UV lamp 10 and is ultimately discharged from outlet 13c. Again, a drain may be provided at or towards the bottom if the inlet is not at the bottom.

In embodiments, even if an inlet is a bottom, it may be found appropriate to also include one or more drains at other locations.

FIG. 6 shows an alternative embodiment in which a plurality of conduits are provided. In this, each one has an inlet/drain at the bottom and moisture passes up the conduit, the conduit being at a position where it lies generally parallel to the UV lamp 10 and spaced therefrom at such a position where UV radiation can effectively enter the conduit and sterilise its contents. The sterilised moisture then outlets at the upper end 13d. Thus, one tube has inlet 12d¹ and outlet 13d¹ another inlet 12d² and 13d² and so on. The conduits will therefore be generally vertically disposed and generally axially parallel to the lamp and around the lamp, typically concentrically. Again, if the units are towards the bottom then no lamp will usually be necessary.

In embodiments where a plurality of conduits are provided, they need not necessarily be straight. They could be curved, helical, have one or more bends or be of any other configuration.

As described, the lamp and conduit (vessel arrangement) may be provided within a housing within the water tank and a schematic example of a non-limiting embodiment of such a housing is shown in FIG. 7. The housing is made of a metal, such as stainless steel or other metal which is generally reflective to UV radiation. The housing 20 comprises a cylindrical base part 21 which receives the conduit (or conduits) and lamp generally axially within it as shown in dashed lines. The housing may be simply formed of side walls having an open base and top or may have closed base and top but with means for allowing ingress and omission of the inlet and/or outlet tube.

A simple helical type conduit is shown but any configuration may be used.

At the top of the housing there is formed an end portion or end cap 22. The and cap is again of UV reflective material such as any suitable metal or stainless steel and includes a top plate 23 which reflects UV rays, side walls 24, and a bottom plate, all walls and end plates being UV reflective. In a preferred embodiment, the end cap includes a first proximal part of generally arcuate, preferably part cylindrical, cross section which extends generally over the cylindrical part of the housing and is shown by 25 and a distal part which includes tapered side walls (in cross section) which extend outward beyond the cylindrical extent of the base housing 21. The outlet, such as 13, of a conduit as shown in any of the embodiments of FIGS. 2 to 6 extends generally within this at a direction which is non-axial to the axial direction of the lamp and helix discharge opening 27 which may extend generally downwardly as shown in FIG. 8, horizontally or upwardly or in any convenient direction for directing the moisture discharge.

Thus, the end portion ensures that UV radiation from the lamp 10 which does not directly impinge upon the parts of the conduit which are wrapped directly round, or directly in contact with, the tube are also affected by the radiation since this is reflected by end cap onto this outlet part 13 of the moisture discharge tube. Particularly the tapering nature of the end part causes the radiation to be reflected and concentrated onto the tube itself, as is shown schematically in FIG. 8 which shows a schematic cross section from the top of the end part and in which the arrows show UV radiation and how this is reflected by the end part, including its tapered distal end 26.

UV radiation is therefore directed to impinge upon the discharge outlet right to the very end or very near to its end and hence most efficiently destroys bacteria and other pathogens, including those which may be found at the very discharge nozzle itself. Of course it will be appreciated that radiation from the UV lamp which are directed at an angle having any non-horizontal component, will be reflected back off the top (upper) wall of the end part and will therefore ultimately be reflected downwardly to the discharge nozzle, or indeed, since the end part also includes a bottom place 28, extending across the bottom part of the end part 22 which extends beyond the cylindrical extent of the housing 21, UV radiation may be reflected from this bottom wall 28 in a direction having a vertical component.

Top plate 23 thus, in some embodiments, extends across the whole end part (cap) wherein bottom plate 28 only extends along the part of this which extends sideways beyond the extent of the cylindrical body. Other embodiments may differ however.

The housing shown in FIG. 7 (or similar housings) will be mounted within the water tank and therefore should be made of a UV reflective material which is robust and substantially unaffected by water, such as stainless steel or other materials.

FIG. 9 shows very schematically a humidifier device in accordance with the present invention in which a UV lamp, a UV transmissive conduit and a reflective housing, all forming a schematically shown component 30 is mounted within a water tank 2 placed upon a body 1. The inlet 12 of any of the embodiments of FIGS. 1 to 6 or other embodiments is mounted so as to receive moisture acted upon by the humidifiers, by ultrasonic means or otherwise and to transmit to be effected by the UV radiation and subsequently discharged through a nozzle as a moisture fog 31.

Embodiments of the invention may also be used with other types of air treatment apparatus such air conditioners, de-humidifiers, etc and it will be apparent how they can be modified for use with such devices. In some devices, the moisture may be discharged at a ‘waste’ outlet, or to the exterior of apparatus or a building, but it an still be important to ensure such moisture is not contaminated.

In other embodiments, water (or other liquid or fluid) within a tank or reservoir 2 may be directly acted upon by UV, eg from one or more UV lamps positioned within the tank.

1. Air treatment apparatus including means for causing ultraviolet radiation to be applied to fluid or moisture which is to be discharged from the apparatus.

2. Apparatus as claimed in claim 1, which is a humidifier, de-humidifier or air conditioning apparatus.

3. Apparatus as claimed in claim 1, comprising a vessel positioned to receive moisture for discharge from the apparatus and a UV generating means such that moisture within the vessel is acted upon by ultraviolet radiation generated by the UV generating means before being discharged.

4. Apparatus as claimed in claim 1, comprising one or more conduits positioned such that moisture within the or each conduit is acted upon by UV generated by the UV generating means, the or each conduit having an outlet from where eradiated moisture is discharged.

5. Apparatus as claimed in claim 4, comprising one or more conduits which are of UV transmissive material.

6. Apparatus as claimed in claim 4, comprising a single conduit.

7. Apparatus as claimed in claim 4, comprising a plurality of conduits.

8. Apparatus as claimed in claim 4, wherein the or each conduit has at least a part which is generally helical.

9. Apparatus as claimed in claim 1, comprising one or more drainage part arranged to allow any water or other liquid which has condensed to drain downwardly.

10. Apparatus as claimed in claim 1, comprising a housing comprising a body of UV reflective material provided around the conduit or conduits or vessel so as to reflect UV radiation back thereto, the UV generator being mountable within the housing.

11. Apparatus as claimed in claim 10, comprising one or more conduits formed of a UV transmissive material, each having an inlet and outlet and positioned so that part of the or each conduit is wrapped around at least part of the UV source and further comprising reflective means for causing UV radiation to be reflected onto one or more parts of the conduit which extend beyond the part which is wrapped around the UV source.

12. Apparatus as claimed in claim 11, comprising means for causing UV radiation to be reflected towards the outlet end of the or each conduit.

13. Apparatus as claimed in claim 1, including a housing for the vessel and/or conduit and UV generating means, the housing including a body part which is generally elongate and has a diameter.

14. Apparatus as claimed in claim 13, wherein the housing includes an end part having a part which extends laterally in at least one direction beyond the diameter of the housing, and in which an outlet part of the or each conduit extends, the end part being adapted to reflect UV radiation from the lamp towards the outlet end.

15. Apparatus as claimed in claim 14, wherein the end part includes one or more tapered reflective portions.

16. Air treatment apparatus substantially as hereinbefore described, with reference to, and as illustrated by claim 1.

17. Sterilising means for moisture to be discharged by an air treatment apparatus, substantially as hereinbefore described with reference to, and as illustrated by, any of the accompanying drawings.
